(19) United States
(12) Patent Application Publication
(10) Pub. No.: US 2026/0024671 A1
(43) Pub. Date: Jan. 22, 2026

(54) Hospital-to-Home Patient Monitoring System

(71) Applicant: OneSource Solutions International, Inc., Sudbury, MA (US)

(72) Inventors: Harold Arkoff, Sudbury, MA (US); Vedran Jukic, 34123 Trieste (TS) (IT)

(73) Assignee: OneSource Solutions International, Inc., Sudbury, MA (US)

(21) Appl. No.: 19/270,393

(22) Filed: Jul. 15, 2025

Related U.S. Application Data
(60) Provisional application No. 63/672,881, filed on Jul. 18, 2024.

(51) Int. Cl. G16H 80/00; G08B 27/00; G16H 10/60; G16H 40/20

(52) U.S. Cl. CPC G16H 80/00; G16H 10/60; G16H 40/20; G08B 27/001

(57) ABSTRACT

The present invention provides a system and method for remotely monitoring discharged hospital patients using Bluetooth-enabled wearable medical devices integrated into a secure, mobile-to-cloud telemetry platform. The system is designed to extend hospital-grade patient observation into residential and non-clinical settings by continuously capturing, transmitting, and evaluating physiological data under clinical supervision. Each patient is outfitted with one or more Bluetooth-enabled sensors configured to measure vital signs such as heart rate, blood pressure, oxygen saturation, temperature, or movement. These devices transmit raw telemetry to a mobile coordination platform that relays encrypted signal data to a centralized hospital dashboard. There, clinicians may review data streams, configure patient-specific thresholds, and receive AI-prioritized deterioration alerts governed by institutional policies.

### CROSS-REFERENCE TO RELATED APPLICATIONS

This application claims the benefit of U.S. Provisional Patent Application No. 63/672,881, filed on Jul. 18, 2024, under 35 U.S.C. § 119(e), the entire contents of which are hereby incorporated by reference. The subject matter disclosed in the provisional application includes the use of Bluetooth-enabled wearable medical devices, a mobile monitoring platform, real-time streaming of vital signs, configuration of threshold alerts, and a discharge-to-home architecture that supports continuous remote patient evaluation.

### BACKGROUND OF THE INVENTION

### Technical Field

The present invention relates generally to medical telemetry systems and, more specifically, to systems and methods for extending hospital-level patient monitoring into home environments using Bluetooth-connected wearable devices. The invention enables discharged patients to remain under continuous clinical supervision through a mobile interface that receives vital sign data from physiological sensors and routes it to hospital-based monitoring dashboards.

Additionally, the invention is designed to accommodate evolving healthcare regulatory requirements (including, but not limited to, HIPAA, GDPR, FDA, and MDR) and is adaptable to future regulatory frameworks. The system is operable with a range of third-party medical devices, health information systems, and telemedicine platforms via open or proprietary APIs, enabling seamless integration with electronic health records (EHR), clinical decision support systems, and remote patient management infrastructures.

Additionally, this invention introduces a new artificial intelligence architecture that operates within a rule-bound and auditable framework, allowing remote alerting decisions to be generated and reviewed under constraint-based grammars and institutional oversight.

### Description of Related Art

Hospitals are increasingly seeking to reduce inpatient load while maintaining continuity of care for patients who are medically stable but still require close physiological monitoring. Conditions such as pneumonia, congestive heart failure, or post-operative recovery often necessitate multi-day observation of vital signs but do not require direct on-site intervention if patients can be safely discharged with remote telemetry. Traditional outpatient follow-up methods, however, fail to provide continuous data and lack mechanisms for timely clinical escalation when deterioration occurs.

Bluetooth-enabled wearable devices provide a low-cost, scalable means of capturing real-time physiological signals, including blood pressure, heart rate, oxygen saturation, temperature, and movement data. These sensors can be paired with a mobile device that aggregates the signals and transmits them securely to a centralized monitoring hub at the originating hospital or affiliated care facility. However, existing implementations are limited by rigid alert thresholds, lack of patient-specific configurability, and inadequate mechanisms for institutional governance or override. In particular, most systems operate on fixed rules that either generate excessive false alerts or fail to respond to subtle patterns of clinical deterioration.

Moreover, when telemetry-based systems attempt to use artificial intelligence for predictive alerts or patient prioritization, they often do so without enforcing policy constraints, tracking override decisions, or enabling legally auditable action paths. In the absence of structured human oversight, AI-generated decisions can introduce risks in regulated clinical environments. Accordingly, there is a need for a Bluetooth-based, hospital-to-home patient monitoring platform that not only transmits physiological signals in real time, but also incorporates a traceable, constraint-governed artificial intelligence system capable of learning from human feedback and supporting post-hoc adjudication of remote clinical decisions.

The present invention provides a system and method for remotely monitoring discharged hospital patients using Bluetooth-enabled wearable medical devices integrated into a secure, mobile-to-cloud telemetry platform. The system is designed to extend hospital-grade patient observation into residential and non-clinical settings by continuously capturing, transmitting, and evaluating physiological data under clinical supervision. Each patient is outfitted with one or more Bluetooth-enabled sensors configured to measure vital signs such as heart rate, blood pressure, oxygen saturation, temperature, or movement. These devices transmit raw telemetry to a mobile coordination platform that relays encrypted signal data to a centralized hospital dashboard. There, clinicians may review data streams, configure alert thresholds, and receive real-time deterioration warnings for patients previously treated in an inpatient setting.

The present invention contemplates that the sensor, medical device, or combination thereof may comprise any apparatus, subsystem, or system component—whether wearable, implantable, portable, stationary, or environmental—configured to acquire, process, or transmit physiological or health-related data from a patient. The communication between these components and other system modules may employ any wired or wireless protocol now known or hereafter developed, including but not limited to Bluetooth, Bluetooth Low Energy (BLE), Wi-Fi, ZigBee, cellular, radiofrequency (RF), infrared, Ethernet, or other proprietary or open standards.

The invention further incorporates a new agentic artificial intelligence framework that allows remote decisions-such as alert prioritization, escalation routing, and patient engagement actions-to be made autonomously while preserving institutional oversight and legal accountability. This framework comprises a layered system that includes an inference engine, a constraint grammar enforcement layer, an override and trace logging module, a closed-loop learning mechanism, and an accountability router that ensures each AI-generated output can be audited and traced to its governing logic and context. By combining Bluetooth-based physiological telemetry with a multi-layered agentic AI engine, the invention enables the safe discharge of moderate-risk patients to home settings while preserving the real-time clinical visibility, control, and legal defensibility required for hospital-grade care.

### BRIEF DESCRIPTION OF THE DRAWINGS

The drawings presented herein show illustrative embodiments of the disclosure. They do not illustrate all embodiments. Other embodiments may be used in addition to or instead of the illustrative embodiments. Details that may be apparent or unnecessary may be omitted to save space or for more effective illustration. Some embodiments may be practiced with additional components or steps and/or without all the components or steps that are illustrated. When the same numeral appears in different drawings, it refers to the same or like components or steps. The drawings are not intended to depict every feature of every implementation nor relative dimensions of the depicted elements and are not drawn to scale.

FIG. 1 illustrates the full system overview, showing Bluetooth medical sensors, a mobile device relay, encrypted telemetry transfer, and hospital dashboard integration.

FIG. 2 shows the mobile application interface for patients, with real-time signal display, compliance status, and provider-configured alerts.

FIG. 3 details the internal structure of the agentic AI system, including the inference engine, constraint grammar module, override logging, feedback core, and accountability router.

FIG. 4 provides an example of an override workflow, showing clinician review and suppression of an automated alert with rationale logging.

FIG. 5 depicts the medical data governance dashboard, allowing constraint editing, log export, model version control, and system auditing.

### DETAILED DESCRIPTION

In the following description, numerous specific details are set forth to clearly describe various specific embodiments disclosed herein. One skilled in the art, however, will understand that the subject matter of the present disclosure may be practiced without all of the specific details discussed below. In other instances, well-known features may not have been described so as not to obscure the invention with unnecessary detail regarding known features.

### Definitions

Bluetooth-enabled wearable medical device: a sensor that measures physiological parameters and transmits wirelessly to a paired device.

Hospital-to-home monitoring system: infrastructure enabling discharged patients to be monitored remotely using real-time data.

Threshold alert configuration: the clinical rule that determines which physiological readings trigger notifications.

Agentic artificial intelligence: AI structured with enforceable rules, override paths, traceability, and feedback control.

Constraint grammar layer: policy-encoded rules that permit or block AI outputs in real-time.

Override logging subsystem: a traceable module for recording human interventions into system decisions.

Closed-loop feedback: the iterative process where human feedback informs future system behavior.

Accountability routing: metadata tagging for downstream traceability and governance.

Sensor: Any component or subsystem configured to detect, measure, or acquire physiological, environmental, or health-related data from a patient.

Medical Device: Any apparatus, system, or component—wearable, implantable, portable, stationary, or environmental—configured to acquire, process, or transmit physiological or health-related data.

Relay Component: Any intermediary device, gateway, network interface, or module configured to receive and forward data between system components.

Monitoring Interface: Any dashboard, user interface, display, or analytical platform configured to present data, alerts, or recommendations to a user.

Audit Log: An immutable or tamper-evident record of data receipt, alert generation, configuration changes, user interactions, and system events.

As used herein, the terms “sensor,” “medical device,” or “combination thereof” refer to any apparatus, subsystem, or system component—whether wearable, implantable, portable, stationary, or environmental—configured to acquire, process, or transmit physiological or health-related data from a patient. Examples include, but are not limited to, wearable sensors, bedside monitors, implantable devices, home-based medical monitoring stations, smart beds, infusion pumps, and any other device capable of capturing and transmitting relevant patient parameters.

The communication between the sensor, medical device, or combination thereof and other system components may employ any wired or wireless protocol now known or hereafter developed, including but not limited to Bluetooth, Bluetooth Low Energy (BLE), Wi-Fi, ZigBee, cellular, radiofrequency (RF), infrared, Ethernet, or other proprietary or open standards. In some embodiments, the system further supports future communication protocols or hybrid combinations thereof.

In some embodiments, the patient monitoring system operates by: receiving telemetry data from patient sensors; generating a candidate alert or recommendation using the agentic artificial intelligence inference engine; validating this output against a constraint grammar encoded in a hospital rule repository; routing outputs that violate constraints to a human override queue for review; accepting input from credentialed reviewers; and storing all override actions with comprehensive audit metadata, including reviewer identity, timestamp, decision context, and model version.

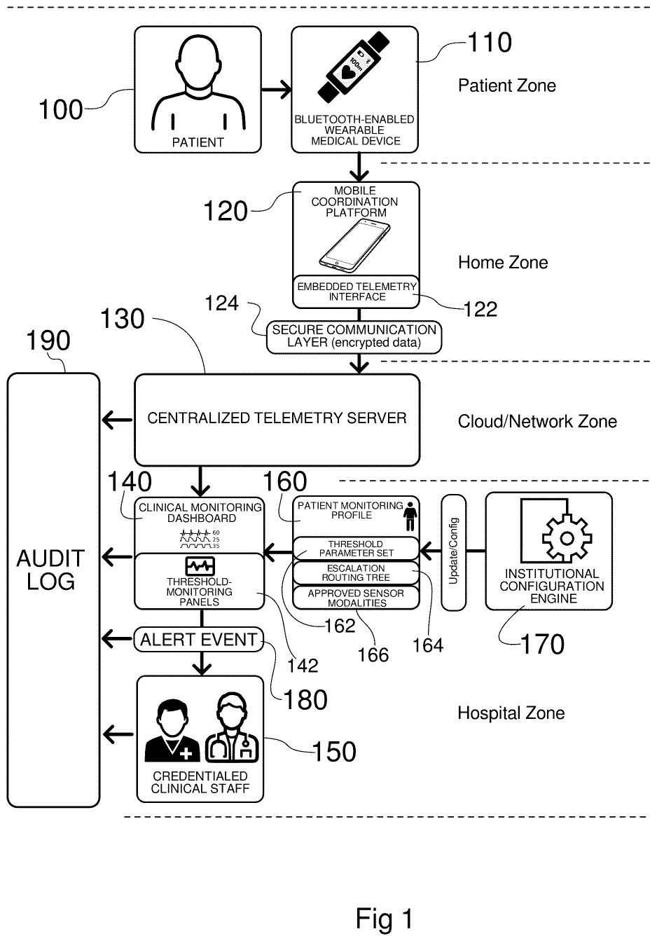

Referring to FIG. 1, each patient (100) is outfitted with one or more Bluetooth-enabled wearable medical devices (110), which may include sensors for heart rate, oxygen saturation, respiratory rate, temperature, and blood pressure. These devices are paired via Bluetooth Low Energy (BLE) protocol to a mobile coordination platform (120), typically implemented as a smartphone or tablet secured by institutional provisioning.

The mobile platform includes an embedded telemetry interface (122) responsible for receiving raw physiological signal data from the sensors 110, validating signal integrity, and applying time-stamping and compression logic. The interface performs local noise filtering and data packet framing prior to outbound transmission. Telemetry is relayed over a secure communication layer (124), which supports both Wi-Fi and 4G/5G cellular fallback, using a hospital-issued encryption certificate for outbound authentication.

The encrypted telemetry is transmitted to a centralized telemetry server (130), which acts as the ingestion point for all patient data. The server maintains persistent data queues, applies ingestion timestamp verification, and routes signal streams to one or more clinical monitoring dashboards (140) associated with the originating care institution. Each dashboard instance displays real-time waveform and threshold-monitoring panels (142), allowing credentialed staff (150) to observe current readings, configure alerts, acknowledge events, and perform interventions.

Each patient is assigned a monitoring profile (160) stored within the institutional configuration engine (170). This profile contains individually specified alert thresholds (162), escalation routing trees (164), and approved sensor modalities (166). Clinicians may update the profile dynamically, and updates propagate to both the mobile platform and the cloud telemetry router in under 5 seconds.

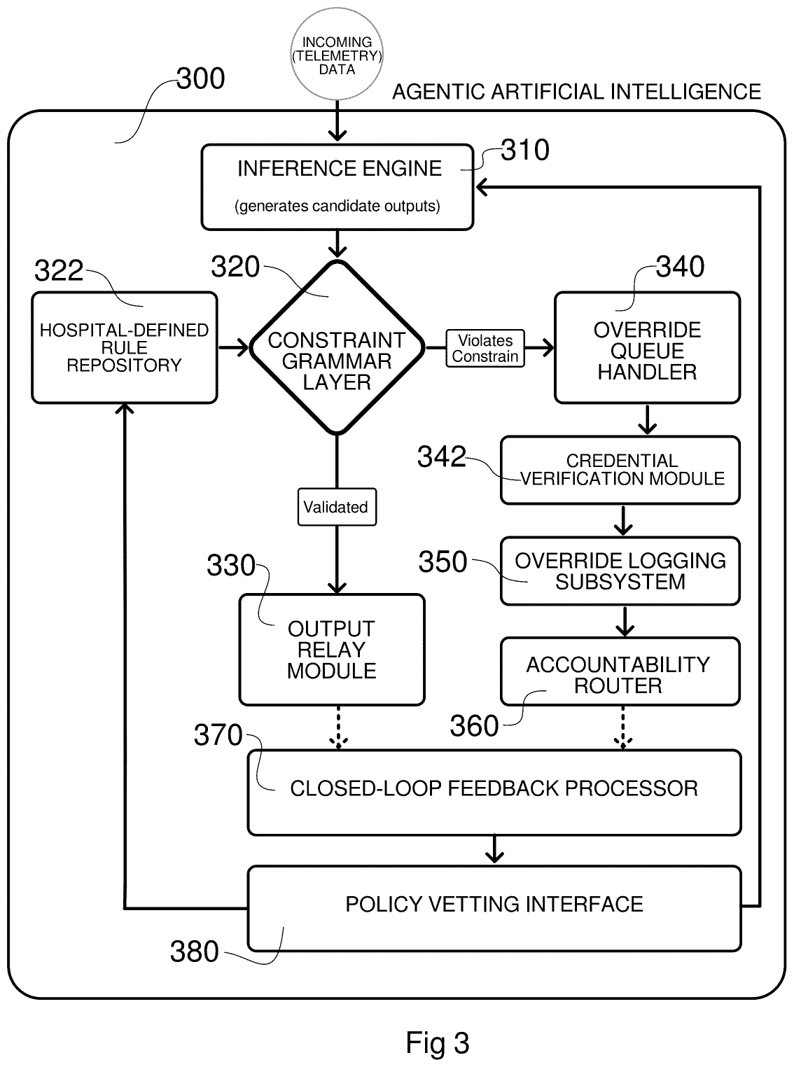

In the event that sensor data crosses a configured threshold, the threshold indicating imminent danger for the patient, the server generates an alert event (180) containing the sensor source, triggering parameter, timestamp, and relevant patient metadata. Alerts are queued for either direct clinician review or agentic AI processing, depending on institutional policy. In configurations that include agentic enforcement (covered in FIGS. 3-5), all alert decisions pass through the AI constraint validation engine before any downstream action is taken.

A complete audit log (190) captures telemetry receipt, alert generation, threshold changes, and dashboard activity. All events are stored with cryptographic integrity markers and can be queried for institutional review or external regulatory inspection.

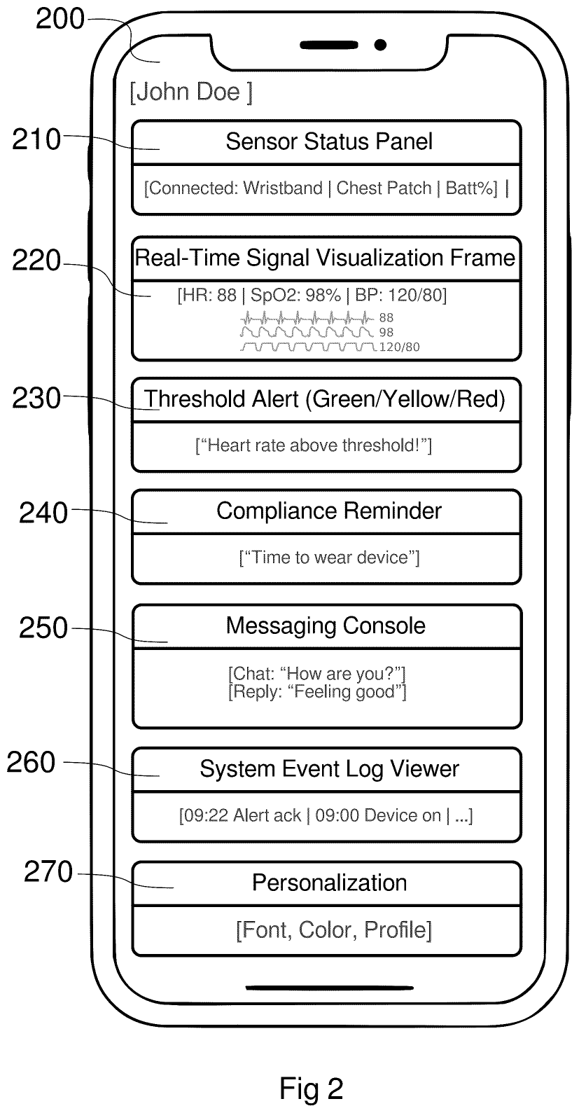

Referring to FIG. 2, the invention includes a mobile application interface designed to facilitate patient-side participation in the hospital-to-home monitoring process. The interface is hosted on the same mobile coordination platform (120) described in FIG. 1 and provides patients with real-time feedback, device management tools, and structured communication from the care team.

Each patient interacts with a dedicated patient interface module (200), which is tailored to their monitoring plan and literacy profile. The primary interface screen includes a sensor status panel (210) that displays the live connectivity state, battery level, and data transmission activity for each paired wearable medical device (110). This allows patients or caregivers to verify that all expected devices are properly attached and operational.

The interface further includes a real-time signal visualization frame (220), which renders patient-specific physiological data, such as pulse rate or oxygen saturation, in an easily interpreted format. Signal values may be displayed as rolling line graphs, color-coded indicators, or discrete value blocks depending on configuration settings. Readings are updated at intervals determined by sensor frequency and transmission latency.

Each monitored parameter is governed by its corresponding threshold alert indicator (230), which provides visual or auditory feedback when a reading approaches or exceeds configured limits. The threshold indicators are synchronized with backend logic in the telemetry server (130) and are dynamically updated when providers modify alert thresholds in the patient's profile (160). Alert tones, vibration cues, or color changes may be triggered locally, accompanied by an instruction prompt or recommendation.

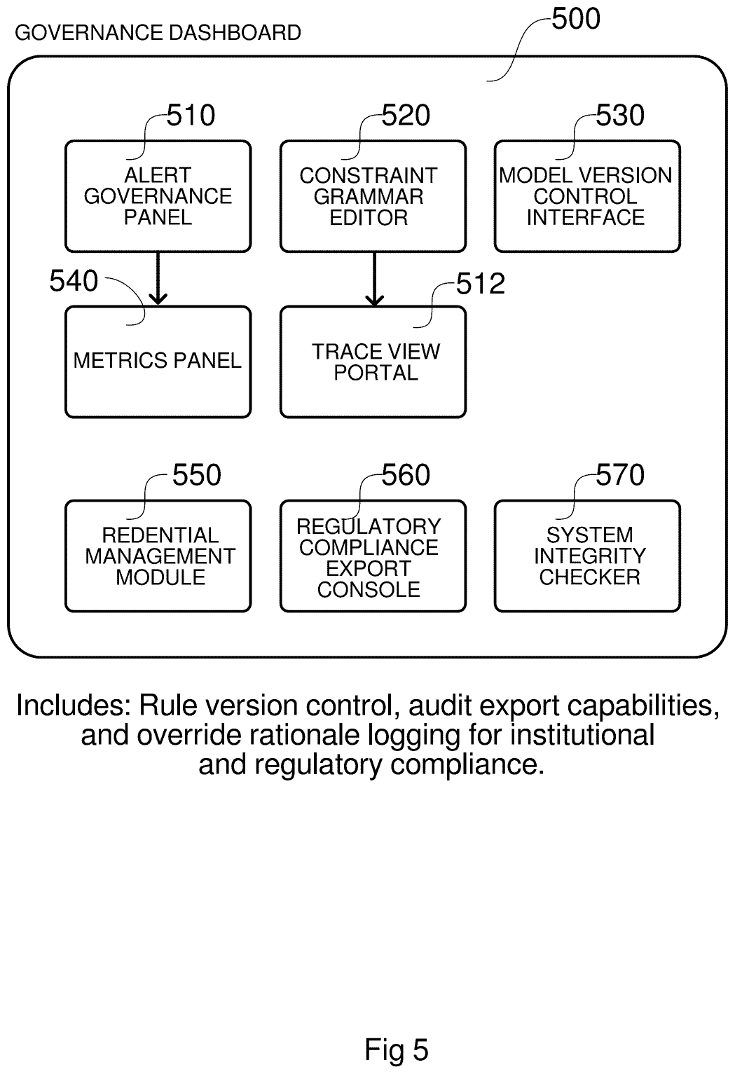

The interface contains a compliance reminder module (240), which presents timed alerts to encourage sensor wear adherence, medication ingestion, or behavioral tracking. These reminders are configured by clinical staff through the governance dashboard (described in FIG. 5) and transmitted to the patient interface through a secure push notification protocol. Reminders may also be acknowledged or postponed, with responses logged for later review.

A provider messaging console (250) is integrated into the interface, allowing two-way communication between the patient and designated staff (150). The console supports structured prompts (e.g., “Are you feeling short of breath?”), free-text response boxes, and predefined action buttons. Messages are timestamped and routed through the clinical dashboard, where escalation pathways may be activated if patient input indicates deterioration or risk.

The mobile application also includes a system event log viewer (260), which displays a record of sensor activity, alerts, acknowledgments, and completed tasks. This viewer enhances transparency for the patient and may assist caregivers in troubleshooting or reporting.

All interface content is rendered through a local personalization layer (270), which adapts fonts, icons, language, and color schemes based on patient literacy level, visual acuity, or institutional branding. The personalization layer supports dynamic theme updates without requiring application reinstall or manual reconfiguration.

Referring to FIG. 3, the invention includes a multi-layered agentic artificial intelligence engine (300) designed to process incoming physiological telemetry, generate clinical decision outputs, and ensure that all automated actions comply with institutional, regulatory, and ethical constraints. This architecture introduces decision-bound AI with human oversight, embedded traceability, and structured override handling.

At its core is the inference engine (310), which receives preprocessed signal streams from the telemetry server (130) and synthesizes them using one or more models trained on physiological trend patterns, historical alert resolution rates, and patient-specific metadata. The engine supports rule-based, statistical, or neural inference depending on deployment configuration and institutional approval.

Each output generated by the inference engine—such as a deterioration alert or escalation recommendation—is passed through a constraint grammar layer (320). This subsystem evaluates the proposed action against a hospital-defined rule repository (322), which encodes local policy restrictions, national regulatory limits, and time-of-day or patient-class modifiers. The grammar operates in a logic tree structure and may include threshold gates, dependency conditions, and multi-parameter suppression logic.

If a candidate output satisfies all constraints, it is routed to the output relay module (330) for transmission to either the hospital dashboard (140) or the mobile platform (120). If the output violates one or more constraints, it is suppressed or redirected to the override queue handler (340), where it is presented for clinician review before execution.

The override queue handler is integrated with a credential verification module (342), which ensures that only authorized personnel—typically attending physicians or registered nurses—may approve or edit suppressed outputs. When a clinician overrides or modifies a system decision, the interaction is recorded in the override logging subsystem (350), which stores metadata including the user ID, decision type, timestamp, rationale code, and override result.

All decisions—executed, suppressed, or modified—are appended with provenance metadata via the accountability router (360). This router attaches structured fields including model version, grammar hash, input trace ID, and override linkage record. The outputs are stored in the institutional audit log (190) and made available through the governance dashboard (described in FIG. 5).

A dedicated closed-loop feedback processor (370) continuously reviews override trends, user feedback, and system error rates. The processor flags high-frequency override patterns for re-evaluation and proposes updates to the inference engine (310) or constraint grammar rules (322) subject to administrative approval. All proposed updates must pass through the policy vetting interface (380), where designated reviewers approve, reject, or defer model or grammar changes.

The agentic AI engine supports both real-time inference and asynchronous decision review and ensures that all system-generated decisions are bounded by policy, logged for traceability, and subject to institutional override.

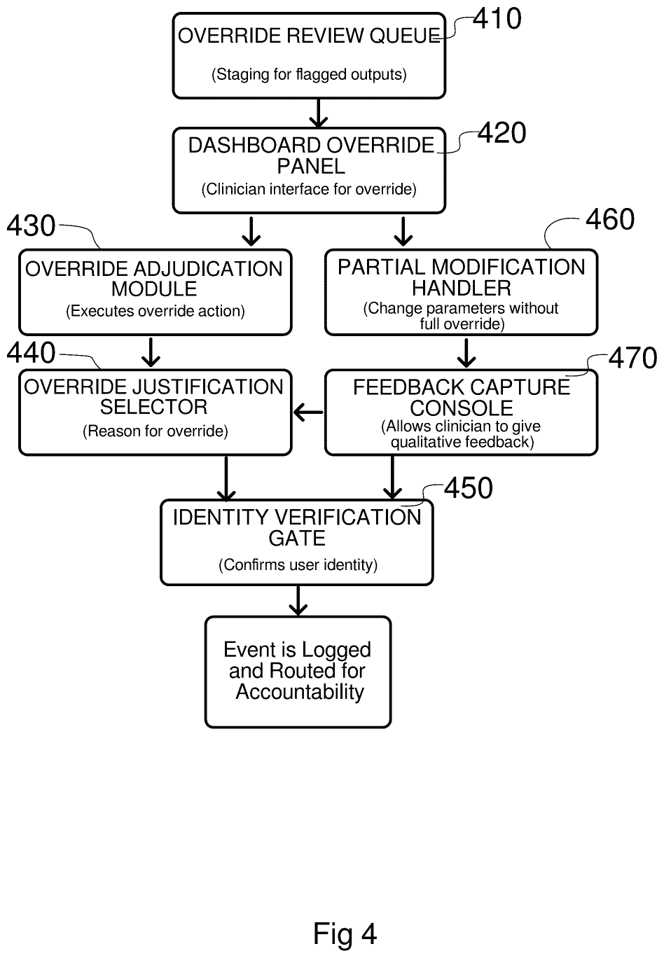

Referring to FIG. 4, the invention includes a formal override mechanism that allows credentialed clinical users to review, suppress, modify, or confirm AI-generated outputs that have been flagged by the constraint grammar layer (320) as potentially policy-infringing or ambiguous. This mechanism ensures human oversight in safety-critical situations and provides a fully traceable decision trail.

In a typical scenario, the AI engine (300) generates an alert based on elevated heart rate and decreasing SpO₂ for a monitored patient. The proposed alert fails a composite safety constraint within the hospital-defined rule repository (322)—for instance, because the patient's prior discharge plan includes acceptable short-term desaturation events. The alert is thus withheld and placed into the override review queue (410).

A clinician accessing the system via the dashboard override panel (420) is presented with the candidate alert, contextual telemetry, recent patient interactions, and the rationale for constraint failure. The clinician may then perform one of several actions: (1) confirm the suppression; (2) override and escalate; or (3) modify and reroute the alert (e.g., assign lower severity or defer to tele-nurse).

All override actions are governed by the override adjudication module (430), which logs the decision event to the override logging subsystem (350) and prompts the user for an accompanying justification note. The system displays a structured override justification selector (440), containing common rationale categories such as “Known baseline deviation,” “Duplicate alert,” “False trigger,” or “Patient under alternate supervision.” A free-text field is also provided.

The override event is time-stamped and authenticated using the identity verification gate (450), which cross-references the user session with the credential verification module (342). Once verified, the decision is relayed to the accountability router (360) and embedded into the patient's telemetry audit log (190).

In cases where an override modifies rather than cancels an alert, the partial modification handler (460) activates, permitting the clinician to change parameters such as alert recipient, color-coding, delivery delay, or severity level. These edits are logged independently of full suppression or escalation events.

Clinicians may also provide feedback about the alert's relevance or usability through the feedback capture console (470). This console includes sliders for signal clarity, contextual accuracy, and perceived urgency, along with optional commentary. Feedback is aggregated by the closed-loop feedback processor (370), which uses it to recommend future adjustments to model sensitivity or grammar constraints.

All override decisions are displayed in real time on the governance dashboard (described in FIG. 5), where institutional administrators can monitor trends, flag outliers, and export decision logs. This system creates a complete, tamper-evident audit trail for every AI decision path that involves human intervention.

Referring to FIG. 5, the invention includes a governance dashboard (500) that serves as the institutional control center for managing, auditing, and refining the behavior of the agentic AI engine (300). This dashboard is accessible only to authorized administrators and policy stewards, and is secured under hospital information governance protocols.

At the core of the dashboard is the alert governance panel (510), which presents real-time statistics about all AI-generated alerts, including their type, origin, delivery latency, and final disposition (executed, suppressed, or overridden). Each record is color-coded by status and is linked to a trace view portal (512) that displays the full provenance of the decision path, including the originating inference, constraint grammar applied, any override action, and downstream result.

The dashboard also includes a constraint grammar editor (520), which allows authorized personnel to view, modify, and version control the rules enforced by the constraint grammar layer (320). Grammar elements may include numeric thresholds, temporal constraints, semantic restrictions, patient-type exclusions, and regulatory bindings. The editor includes guardrails to prevent unsafe modifications and supports pre-deployment simulation of impact on historical alert data.

A model version control interface (530) provides access to all prior and current versions of the inference engine (310), including change logs, update rationales, and activation timestamps. This interface is linked to the policy vetting interface (380), allowing newly proposed model or grammar changes from the feedback processor (370) to be reviewed and either approved, deferred, or rejected.

The dashboard also includes a metrics panel (540), which displays institution-wide override rates, most commonly triggered grammar rules, clinician feedback scores, and alert suppression frequency. These metrics are visualized in graphs, tables, and heatmaps, and are used by administrators to detect drift, over-alerting, under-reporting, or inappropriate override trends.

A credential management module (550) controls which users may view, modify, or approve system-level changes. Role-based access tiers are enforced, and all configuration changes are logged via the accountability router (360) and stored in the audit log (190). Credential rotation and access expiration policies are also enforced.

The dashboard supports full export of all logs, grammar rule sets, and override metadata through the regulatory compliance export console (560). This console ensures that institutions can satisfy external audit, litigation, or accreditation requirements by generating machine-readable reports with embedded provenance chains and attestation markers.

Lastly, the system integrity checker (570) continuously scans for configuration anomalies, unreviewed feedback items, rule-version mismatches, or suspended alerts, and raises administrative alerts when intervention is needed. This ensures continuous oversight and operational safety.

In certain embodiments, the system supports remote or automated software updates, reconfiguration, or security patching of system components to maintain clinical, security, or regulatory compliance. The relay component or local device may be configured to perform preliminary signal processing, data validation, or alert generation in environments with intermittent or limited connectivity, ensuring reliable operation in diverse care settings.

System components may be accessed or configured by clinical and non-clinical users, including but not limited to patients, caregivers, technical support personnel, or administrative staff, subject to appropriate security and audit controls. Role-based access ensures that configuration and monitoring privileges are restricted to authorized individuals.

### Artificial Intelligence Engine Variations

As used in this disclosure, the term “artificial intelligence engine,” “AI engine,” or “automated decision engine” encompasses any system or component configured to analyze patient data, generate alerts, or recommend actions based on such data. While in some embodiments the AI engine is implemented as an “agentic” artificial intelligence—characterized by enforceable rules, constraint grammars, override mechanisms, and traceable decision paths—in other embodiments the AI engine may include, but is not limited to, traditional rule-based systems, expert systems, fuzzy logic, machine learning algorithms, deep learning networks, statistical models, ensemble methods, or any combination of automated decision-making technologies. The invention therefore is not limited to a particular type or architecture of artificial intelligence.

While several illustrative embodiments of the present disclosure have been shown and described, numerous variations and alternative embodiments will occur to those skilled in the art. Such variations and alternative embodiments are contemplated and can be made without departing from the scope of the disclosure as defined in the appended claims.

As used in this specification and the appended claims, the singular forms “a,’‘an,” and “the” include plural referents unless the content clearly dictates otherwise. The term plurality” includes two or more referents unless the content clearly dictates otherwise. Unless defined otherwise, all technical and scientific terms used herein have the same meaning as commonly understood by one of ordinary skill in the art to which the disclosure pertains.

The foregoing detailed description is provided to illustrate various embodiments of the invention and is not intended to be limiting. Modifications and substitutions by those skilled in the art are considered within the scope of the invention as set forth in the appended claims. Unless otherwise indicated, all terms used herein have the same meaning as commonly understood by one of ordinary skill in the art to which this invention pertains. The singular forms “a,” “an,” and “the” include plural referents unless the context clearly dictates otherwise. The terms “comprising,” “including,” and similar expressions are open-ended and intended to encompass additional elements, steps, or features not expressly recited. All cited references and incorporated materials are intended to be part of the disclosure to the extent permissible by law.

1-20 (canceled)

21. A hospital-to-home patient monitoring system comprising:
a plurality of medical sensors configured to capture physiological parameters of a patient;
a relay device configured to transmit sensor data over a secure network;
a monitoring server comprising a processor and a memory storing executable instructions that, when executed, cause the processor to:
receive the sensor data;
generate a preliminary clinical recommendation using an inference engine;
validate the clinical recommendation against a policy rule repository storing constraint rules;
route non-compliant recommendations to an override queue accessible by credentialed clinicians; and
log decision metadata including timestamp, patient identifier, clinician identifier, policy rule identifier, and AI model version;
a clinician interface configured to display validated and overridden recommendations and to update a patient record in real time.

22. A patient monitoring system comprising:
a plurality of networked monitoring devices generating sensor telemetry;
a distributed policy repository stored across a plurality of servers;
a governance module comprising:
a validation engine configured to compare candidate AI-generated recommendations against the constraint rules; and
a synchronization module that distributes approved policy rule updates across the plurality of servers;
an audit subsystem configured to store validation results in a secure audit log.

23. The system of claim 21, wherein the relay device comprises a mobile application operable on a smartphone or tablet.

24. The system of claim 21, wherein the constraint rules comprise temporal rules governing escalation of alerts.

25. The system of claim 21, wherein the inference engine comprises a neural network trained on patient telemetry data.

26. The system of claim 22, wherein the synchronization module transmits policy updates using HL7 or FHIR resources.

27. The system of claim 22, wherein the audit log comprises a secure, append-only data structure.

28. The system of claim 22, wherein the validation engine discards stale recommendations that fail timeliness thresholds.

29. A method for governance of AI-driven patient monitoring, comprising:
receiving sensor telemetry from a plurality of monitoring devices;
generating, by an inference engine executed by a processor, a clinical recommendation;
validating the clinical recommendation using constraint rules stored in a policy repository;
routing non-compliant recommendations to an override queue for clinician review;
receiving override input from the clinician;
updating a patient record with the validated or overridden recommendation; and
logging metadata comprising clinician identifier, policy rule identifier, override rationale, and model version information.

30. The method of claim 29, wherein the telemetry is encrypted prior to transmission.

31. The method of claim 29, wherein compliance reports are generated and exported to a regulatory authority.

32. The method of claim 29, wherein override outcomes are aggregated to propose updates to the constraint rules.

33. The method of claim 29, wherein policy updates are distributed across multiple institutions.

34. The method of claim 29, wherein override actions are stored in a secure audit trail.

35. The method of claim 29, further comprising retraining the inference engine using override outcomes.

36. A non-transitory computer-readable medium storing instructions that, when executed by a processor, cause the processor to perform operations comprising:
receiving patient sensor data from a plurality of monitoring devices;
generating a clinical recommendation using an inference engine;
validating the recommendation against constraint rules stored in a policy repository;
routing invalid recommendations to a clinician override queue;
receiving override instructions from a credentialed clinician; and
recording decision metadata including timestamp, patient identifier, clinician identifier, policy rule identifier, and AI model version in an audit log.

37. A non-transitory computer-readable medium storing instructions that, when executed by a processor, cause the processor to perform operations comprising:
receiving patient telemetry data;
generating candidate recommendations using an inference engine;
validating the candidate recommendations against constraint rules stored in a policy repository;
discarding recommendations that fail validation; and
logging validation outcomes with model version identifiers in an audit record.

38. The non-transitory computer-readable medium of claim 36, wherein the instructions further cause the processor to retrain the inference engine using override outcomes.

39. The non-transitory computer-readable medium of claim 36, wherein the audit log is exportable in compliance with healthcare regulatory standards.

40. The non-transitory computer-readable medium of claim 37, wherein validation outcomes include policy rule identifiers.

41. The non-transitory computer-readable medium of claim 37, wherein the audit record is a secure, append-only structure.

42. The non-transitory computer-readable medium of claim 37, wherein validation is performed within a real-time threshold to enable alarm escalation.

43. The non-transitory computer-readable medium of claim 37, wherein policy updates are synchronized across multiple institutions.
